(19) United States
(12) Patent Application Publication
(10) Pub. No.: US 2018/0344190 A1
(43) Pub. Date: Dec. 6, 2018

(54) ELECTROCARDIOGRAM-ASSISTED INSTRUMENTATION AND METHODS OF USING THE SAME

(71) Applicant: UBER DIAGNOSTICS PTE. LTD., Singapore (SG)

(72) Inventors: Ashim Roy, Bangalore (IN); Govind Rajagopalan, Bangalore (IN); Avin Agarwal, Bangalore (IN)

(21) Appl. No.: 16/055,694

(22) Filed: Aug. 6, 2018

Related U.S. Application Data
(63) Continuation of application No. PCT/IB2017/050100, filed on Jan. 10, 2017.

(30) Foreign Application Priority Data
Jan. 13, 2016 (IN) 201621001298

(51) Int. Cl. A61B 5/0456; A61B 5/0472; A61B 5/04

(52) U.S. Cl. CPC A61B 5/0456; A61B 5/02028; A61B 5/04012; A61B 5/0472

(57) ABSTRACT

Medical devices are travel in arteries or veins with an electrical sensor to detect heart nearness. A computer-processor-based system receives and analyzes the sensor signals to provide accurate position of the device based on one or more electrical characteristics. A P-wave and an R-wave of a heartbeat may be extracted from the signals. Energy of the P-wave, the slope of the P wave, and/or a comparison of highest values of the P and R waves can be provided, potentially over time as the device is advanced. A graphical interface may display these values, a speaker may provide pings or beeps associated with values, or any other output that can be sensed may be used. Thresholds associated with reaching the heart may trigger notifications when detected in the sensor signals, such as maximum energy of a P-wave over time, inversion of slope of the P-wave, and/or P-wave amplitude equaling R-wave amplitude.

### RELATED APPLICATIONS

This application claims priority under 35 U.S.C. § 120 to, and is a continuation of, co-pending International Application PCT/IB2017/050100, filed Jan. 10, 2017 and designating the US, which claims priority to Indian Application 201621001298, filed Jan. 13, 2016, such Indian Application also being claimed priority to under 35 U.S.C. § 119. These Indian and International applications are incorporated by reference herein in their entireties.

### BACKGROUND

The heart is a muscular organ in humans and other animals that pumps blood through the blood vessels of the circulatory system. As shown in FIG. 2, in humans, other mammals, and birds, heart 4 is divided into four chambers: upper left and right atria; and lower left and right ventricles. A cardiac cycle refers to a complete heartbeat from its generation to the beginning of the next beat, and so includes the diastole, the systole, and the intervening pause. The frequency of the cardiac cycle is described by the heart rate, which is typically expressed as beats per minute. A normal rhythmical heartbeat, called a sinus rhythm, is established by the sinoatrial node, the heart's pacemaker. An electrical signal is created that travels through the heart 4, causing the heart muscle to contract. Each electrical signal begins in a group of cells called the sinus node or sino-atrial (SA) node 41. As shown in FIG. 2A, SA node 41 is located in the right atrium, which is the upper right chamber of the heart. In a healthy adult heart 4 at rest, the SA node 41 sends an electrical signal to begin a new heartbeat 60 to 100 times a minute.

As shown in FIG. 1, electrocardiography (ECG or EKG, herein referred to as ECG) 1 is the process of recording the electrical activity of a heart over a period of time using electrodes 5 placed on a patient's body. Electrodes 5 detect tiny electrical changes on the skin that arise from the heart muscle depolarizing during each heartbeat. In FIG. 1, a patient's heart activity is being recorded using electrocardiogram. As electrical changes progress from the SA node 41 (FIG. 2A), the signal travels through the right and left atria. This causes the atria to contract, which helps move blood into the heart's lower chambers, the ventricles. The electrical signal moving through the atria is recorded as P-wave on the machine called an electrocardiogram (also ECG or EKG, herein referred to as EKG) 2 performing electrocardiography. The electrical signals are picked up by electrodes 5 and delivered to EKG 2 via electrical connections 6.

FIG. 3 is a detailed illustration of a typical ECG 1 showing a repeating cycle of three electrical entities: a P-wave (atrial depolarization), a QRS complex (ventricular depolarization), and a T-wave (ventricular repolarization). The ECG 1 is traditionally interpreted methodically to catch important findings. As the electrical signal passes through the heart, it moves between the atria and ventricles through a group of cells called the atrio-ventricular (AV) node 42 (FIG. 2A). The signal slows down as it passes through the AV node 42. This slowing allows the ventricles enough time to finish filling with blood. On ECG 1, this part of the process is the flat line between the end of the P-wave and the beginning of the Q-wave. The electrical signal then leaves the AV node 42 and travels along a pathway called the bundle of His 43. From there, the signal travels into the right and left bundle branches. The signal spreads quickly across the heart's ventricles, causing them to contract and pump blood to the lungs and the rest of the body. This process is recorded as the QRS waves on the ECG 1. The ventricles then recover their normal electrical state (shown as the T-wave on the ECG). The muscle stops contracting to allow the heart to refill with blood. This entire process continues over and over with each new heartbeat.

In ECG 1, the P-wave represents depolarization of the atria. Atrial depolarization spreads from the SA node 41 towards the AV node 42, and from the right atrium to the left atrium. The P-wave is typically upright in most leads except for a VR; an unusual P-wave axis (inverted in other leads) can indicate an ectopic atrial pacemaker. If the P-wave is of unusually long duration, it may represent atrial enlargement. Typically, a large right atrium gives a tall, peaked p-wave while a large left atrium gives a two-humped bifid P-wave. The P-wave duration is less than 80 ms.

In ECG 1, the PR interval is measured from the beginning of the P-wave to the beginning of the QRS complex. This interval reflects the time the electrical impulse takes to travel from the sinus node through the AV node 42. A PR interval shorter than 120 ms suggests that the electrical impulse is bypassing the AV node 42 as in Wolf-Parkinson-White syndrome. A PR interval consistently longer than 200 ms diagnoses first degree atrioventricular block. The PR segment (the portion of the tracing after the P-wave and before the QRS complex) is typically completely flat but may be depressed in pericarditis. The PR interval is 120 to 200 ms.

In ECG 1, the QRS complex represents the rapid depolarization of the right and left ventricles. The ventricles have a large muscle mass compared to the atria, so the QRS complex usually has a much larger amplitude than the P-wave. If the QRS complex is wide (longer than 120 ms) it suggests disruption of the heart's conduction system, such as in LBBB, RBBB, or ventricular rhythms such as ventricular tachycardia. Metabolic issues such as severe hyperkalemia, or TCA overdose can also widen the QRS complex. An unusually tall QRS complex may represent left ventricular hypertrophy while a very low-amplitude QRS complex may represent a pericardial effusion or infiltrative myocardial disease. The QRS interval is 80 to 100 ms.

In ECG 1, the J-point is the point at which the QRS complex finishes and the ST segment begins. The J point may be elevated as a normal variant. The appearance of a separate J wave or Osborn wave at the J point is pathognomonic of hypothermia or hypercalcemia. The J point may be elevated as a normal variant. The appearance of a separate J wave or Osborn wave at the J point is pathognomonic of hypothermia or hypercalcemia.

In ECG 1, the ST segment connects the QRS complex and the T-wave; it represents the period when the ventricles are depolarized. It is usually isoelectric but may be depressed or elevated with myocardial infarction or ischemia. ST depression can also be caused by LVH or digoxin. ST elevation can also be caused by pericarditis, Brugada syndrome, or can be a normal variant (J-point elevation). The ST segment interval is 160 ms.

In ECG 1, the T-wave represents the repolarization of the ventricles. It is generally upright in all leads except aVR and lead V1. Inverted T-waves can be a sign of myocardial ischemia, LVH, high intracranial pressure, or metabolic abnormalities. Peaked T-waves can be a sign of hyperkalemia or very early myocardial infarction.

In ECG 1, the QT interval is measured from the beginning of the QRS complex to the end of the T-wave. Acceptable ranges vary with heart rate, so it must be corrected by dividing by the square root of the distance between successive R peaks (RR interval). A prolonged QT interval is a risk factor for ventricular tachyarrhythmias and sudden death. Long QT can arise as a genetic syndrome, or as a side effect of certain medications. An unusually short QT can be seen in severe hypercalcemia. The QT interval is less than 440 ms.

In an ECG, a U wave is hypothesized to be caused by the repolarization of the interventricular septum. It normally has a low amplitude, and even more often is completely absent. If the U wave is very prominent, suspect hypokalemia, hypercalcemia or hyperthyroidism.

### SUMMARY

Example embodiments include healthcare implement systems that are moved in arteries or veins, such as catheters and the like, equipped with an electrical detector or other sensor that picks up electrical signals from the heart as the implement is moved toward the heart. The sensor may be at a farthest end of the instrument to best correlate detected electricity with farthest position of the instrument, or at another operative position. A configured electrocardiogram machine receives and processes the signals from the sensor to provide accurate position of the instrument with regard to the heart. This analysis may consider multiple independent aspects of the heart as determined from the electrical signals. For example, a P-wave and an R-wave of a heartbeat may be independently extracted from the signals, and aspects like the overall energy contained in the P-wave, the slope of the P wave, and/or a comparison of the highest values of the P and R waves can be separately provided. These aspects, over time as the instrument is navigated through the blood vessel, indicate where the instrument is with regard to the heart and when the instrument has reached the heart. These values may be provided over time, such as on a visual display charting the aspect values over time as the instrument is advanced or through a speaker providing pings or beeps associated with values. The machine may also analyze the information extracted from the signals for thresholds associated with reaching the heart, such as a maximum overall energy of a P-wave over time, an inversion of a slope of the P-wave, and/or P-wave amplitude equaling R-wave amplitude, and provide an alert when such thresholds are reached. In this way an operator may accurately know the position of an instrument travelling through blood vessels with respect to the heart directly based on electrical information of the heart as received from the instrument itself.

### BRIEF DESCRIPTIONS OF THE DRAWINGS

Example embodiments will become more apparent by describing, in detail, the attached drawings, wherein like elements are represented by like reference numerals, which are given by way of illustration only and thus do not limit the example embodiments herein.

FIG. 1 is an illustration of a related art electrocardiogram system.

FIG. 2 is an illustration of a human heart electrical system.

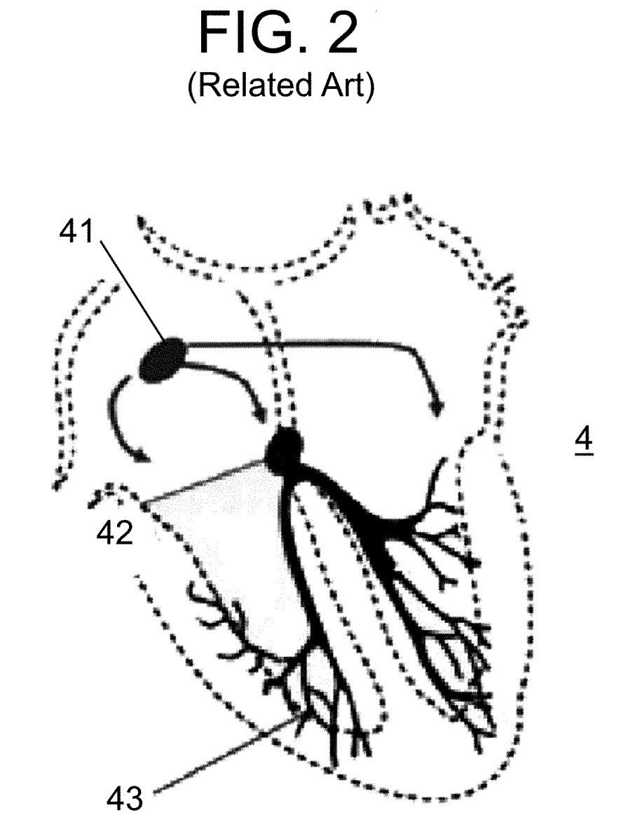

FIG. 3 is an illustration of an ECG of a heartbeat electrical signal.

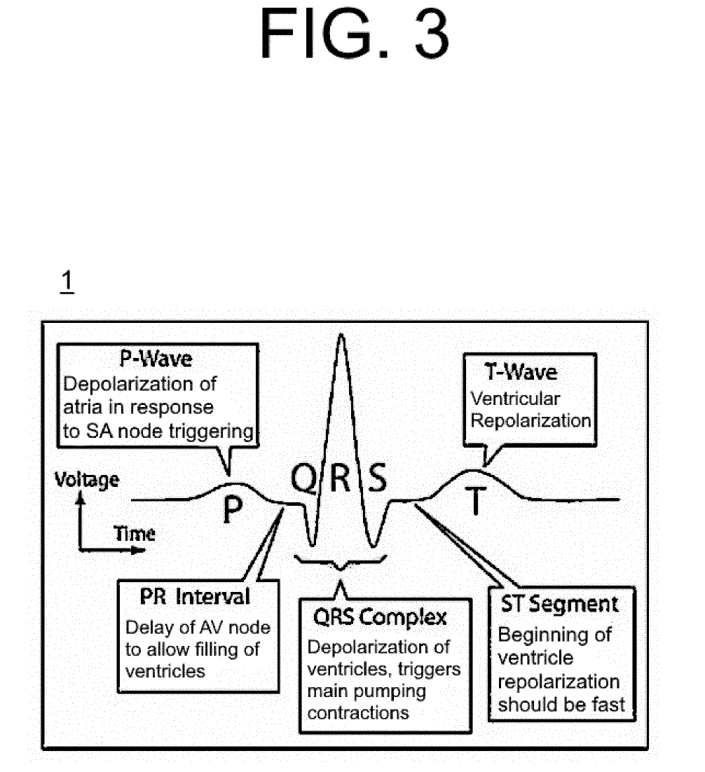

FIG. 4 is an illustration of an example embodiment electrocardiogram system.

FIG. 5 is an illustration of a display output of an example method.

### DETAILED DESCRIPTION

Because this is a patent document, general broad rules of construction should be applied when reading it. Everything described and shown in this document is an example of subject matter falling within the scope of the claims, appended below. Any specific structural and functional details disclosed herein are merely for purposes of describing how to make and use examples. Several different embodiments and methods not specifically disclosed herein may fall within the claim scope; as such, the claims may be embodied in many alternate forms and should not be construed as limited to only examples set forth herein.

It will be understood that, although the terms first, second, etc. may be used herein to describe various elements, these elements should not be limited to any order by these terms. These terms are used only to distinguish one element from another; where there are “second” or higher ordinals, there merely must be that many number of elements, without necessarily any difference or other relationship. For example, a first element could be termed a second element, and, similarly, a second element could be termed a first element, without departing from the scope of example embodiments or methods. As used herein, the term “and/or” includes all combinations of one or more of the associated listed items. The use of “etc.” is defined as “et cetera” and indicates the inclusion of all other elements belonging to the same group of the preceding items, in any “and/or” combination(s).

It will be understood that when an element is referred to as being “connected,” “coupled,” “mated,” “attached,” “fixed,” etc. to another element, it can be directly connected to the other element, or intervening elements may be present. In contrast, when an element is referred to as being “directly connected,” “directly coupled,” etc. to another element, there are no intervening elements present. Other words used to describe the relationship between elements should be interpreted in a like fashion (e.g., “between” versus “directly between,” “adjacent” versus “directly adjacent,” etc.).

As used herein, the singular forms “a,” “an,” and “the” are intended to include both the singular and plural forms, unless the language explicitly indicates otherwise. It will be further understood that the terms “comprises,” “comprising,” “includes,” and/or “including,” when used herein, specify the presence of stated features, characteristics, steps, operations, elements, and/or components, but do not themselves preclude the presence or addition of one or more other features, characteristics, steps, operations, elements, components, and/or groups thereof. The use of “about” in connection with values indicates effective approximation, and such values may vary within a range having substantially similar activity or functionality. As such, values referred to as “about” include similar values and precisions expected with applicable manufacturing tolerances and unavoidable impurities in the element of the value, and generally would be expected to vary less than 15% of the value itself.

The structures and operations discussed below may occur out of the order described and/or noted in the figures. For example, two operations and/or figures shown in succession may in fact be executed concurrently or may sometimes be executed in the reverse order, depending upon the functionality/acts involved. Similarly, individual operations within example methods described below may be executed repetitively, individually or sequentially, so as to provide looping or other series of operations aside from single operations described below. It should be presumed that any embodiment or method having features and functionality described below, in any workable combination, falls within the scope of example embodiments.

The Inventor has recognized a need for successfully navigating a catheter and positioning the tip of the catheter during central venous access. Non-invasive procedures, X-ray, and ultrasound are the most commonly deployed techniques for navigating a catheter. Invasive procedures include fluoroscopy combined with use of electrocardiography (ECG) during catheterization. However, the accuracy of these procedures is variable and depend on attenuated or indirect heart information to guide positioning near the actual heart. The Inventor has developed example embodiments and methods described below to address these and other problems recognized by the Inventor with unique solutions enabled by example embodiments.

The present invention is systems and methods of cardiac instrumentation location. In contrast to the present invention, the few example embodiments and example methods discussed below illustrate just a subset of the variety of different configurations that can be used as and/or in connection with the present invention.

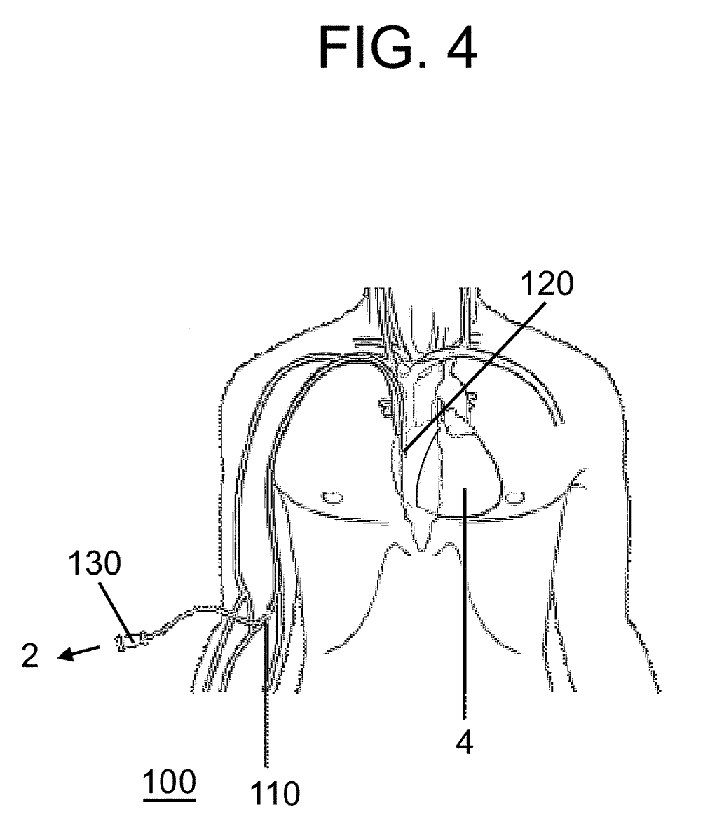

FIG. 4 is an illustration of an example embodiment ECG system 100 and EKG method for accurately navigating a catheter and locating the tip of the catheter during vascular access. As shown in FIG. 4, catheter 110 is inserted into a patient and may be, for example, a peripherally inserted central catheter (PICC) or central venous catheter (CVC). Sensor 120 is positioned at the operative distal tip of a catheter. When catheter 110, such as a PICC or CVC, is inserted in the body, tip sensor 120 of the catheter also captures electrical activity of heart 4. Sensor 120 provides an output of a sensed signal with various components.

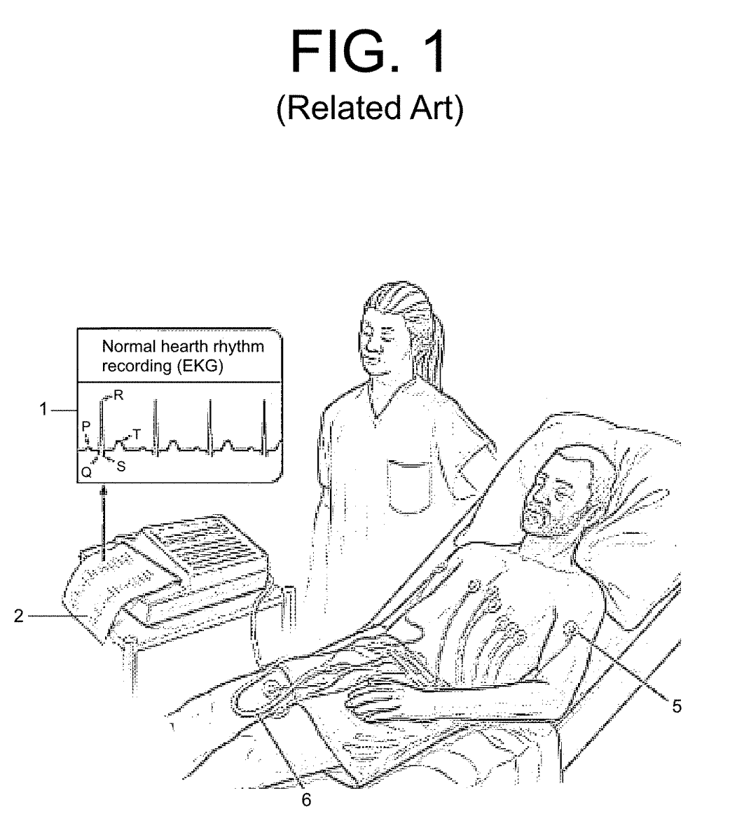

SA node 41 may be considered the epicenter of heart electrical activity because it is a generator of electrical energy. During a typical cardiovascular examination of a patient, surface ECG electrodes 5 (FIG. 1) are connected to the various locations in the body. Electrical energy from SA node 41 is attenuated significantly when it reaches surface electrodes 5. Catheter 110 and sensor 120 provide a lower-resistance path to capture the electrical energy as an ECG signal, because the distal tip of catheter 110 bearing sensor 120 approaches close to heart as catheter 110 is inserted in the body. This sensed signal may be used to accurately navigate and place catheter 110. Signal receiver 130 may be communicably coupled with, or a part of, ECG machine 2 (FIG. 1), which is further configured with example methods discussed below in example embodiment system 100. The ECG signal can thus be analyzed and/or visualized on ECG machine 2 configured to display the signal from receiver 130.

The electrical energy from SA node 41 may slightly attenuate before it reaches the tip of catheter 110. To determine progress of catheter 110 toward SA node 41, three parameters may be analyzed: 1) energy captured at the tip of catheter 110; 2) rate of change of voltage of the P-wave; and 3) the ratio of the P-wave peak and the R-wave peak. These values may be determined from the signal picked up by sensor 120 through receiver 130 and/or EKG machine 2 configured to do so. The energy level at the tip of catheter 110 may be a locus of values that represents or forms a P-wave in an ECG signal. This P-wave—particularly its amplitude—may be extracted and/or stored from sensor 120 for use by example embodiment system 100. Similarly, another locus of values may represent or form an R-wave in an ECG signal. This R-wave—particularly its amplitude—may be extracted and/or stored from sensor 120 for use in system 100. The third parameter, ratio of P-to-R-wave, may be calculated from these amplitudes of the sensed signal. The second parameter, voltage change rate, may extracted and/or stored from the slope of the P-wave.

An energy level may also be extracted from the sensed signal and stored for use by example embodiment system 100. A processor, such as a specific hardware microprocessor with memory and bus, may be configured with appropriate physical structure or executed software to compute a ratio of amplitudes of the P-wave and R-wave from the signal from sensor 120. This computed ratio may be stored and/or used as sets of values of ratios of P-wave and R-wave amplitudes. Additional value sets of the energy of the P-wave over a particular time period and of values of the slope of P-wave may be stored and/or used by the processor. This signal analysis and processing may take place in receiver 130 and/or machine 2 or another separate computer in system 100.

The processor may then aid accurate navigation and locating of catheter 110 with example embodiment system 100 using these data sets: Energy (E), an average energy of the P-wave over several seconds; Slope (S), a rate of change of voltage of the P-wave; and Ratio (R), a ratio of P-wave amplitude and R-wave. In example embodiment system 100, the processor and machine 2 may be configured to display these values in a visual guide for navigating catheter 110, such as on a screen display or printed display. These values may be provided audibly in system 100, such as in the form of an audio guidance beep. In this way, computed values of E, S, and R together may provide operators, such as doctors, with a catheterization guide.

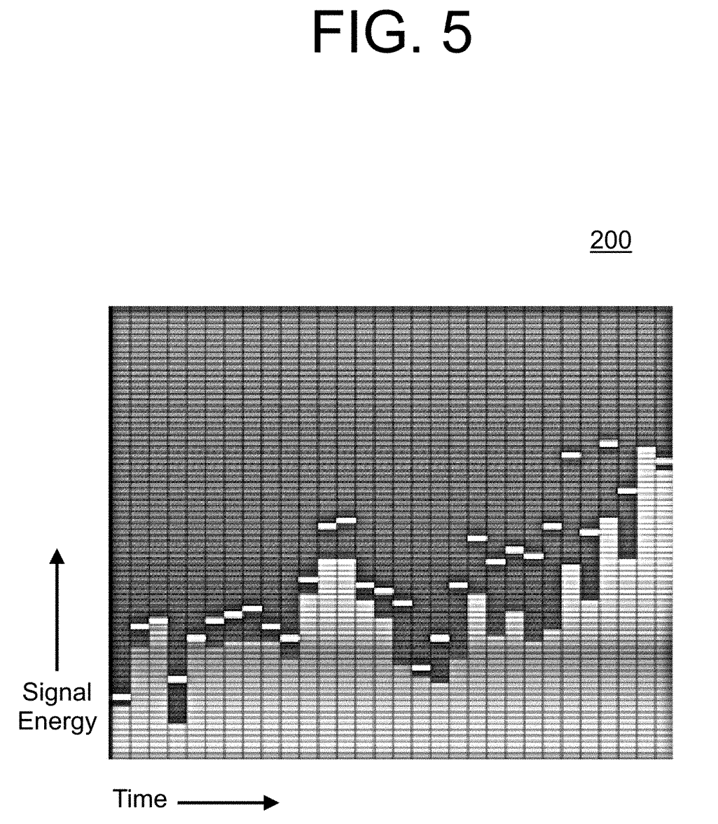

FIG. 5 is an illustration of an example embodiment display 200 showing a graphical visual aid that may be used in combination with audible signals, both of which may be provided on configured machine 2 in example embodiment system 100. Display 200 provides average energy level of each sample (taken over several seconds), with each bar representing the energy for one sample. The time scale is from left-to-right. As catheter 110 approaches SA node 41, the energy captured by sensor 120 at the tip of catheter 110 increases, as shown in later times of FIG. 5. If catheter 110 deviates and moves in a different direction away from SA node 41, the energy captured goes down and the resultant bar size decreases. Similarly, an auditory signal may be produced by machine 2. For example, as a tip of catheter 110 approaches SA node 41, an audio guidance beep may start at a low frequency and increase in frequency and amplitude as the electrical signals indicates that an energy level or other measured parameter indicates nearness to SA node 41. When the tip of catheter 110 has reached a destination point, the audio guidance beep may stay on continuously. P-to-R ratio and P-wave slope may be similarly displayed versus time on display 200.

Energy of the P-wave is always positive and increases as the catheter gets closer to SA node 41. Initially, as catheter 110 is moving toward heart 4, P-wave energy is a monotonically increasing function. Once catheter 110 reaches a point closest to heart 4, there is a sudden drop in energy level of P-wave.

In addition to overall energy of signal detected by sensor 120, slope of the P-wave and P-to-R-wave ratio may be used. The slope of the P-wave is positive when catheter 110 is moving closer to SA node 41. If catheter 110 goes past SA node 41, the slope changes from positive to negative. That is, initially, the slope is monotonically increasing, as catheter 110 is moving towards the heart, and then when catheter 110 is located closest to heart 4, there is sudden change from positive slope to negative slope.

The ratio of P and R-wave amplitude is initially very small when catheter 110 is inserted and then increases as catheter moves closer to SA node 41. The ratio comes very close to 1 at SA node 41. When catheter 110 is advanced in to a patient, the P-wave to R-wave amplitude ratio is a monotonically increasing function. This behavior stops when catheter 110 has reached the closest point to heart 4. At this point, the P-wave to R-wave amplitude ratio falls suddenly.

Energy level and these inflections of the slope of the P-wave and ratio of P and R-wave approaching unity may be used as visual and auditory indicators, the same way as energy described above, or as thresholds for alarming or increasing notifications from machine 2.

As seen, because as catheter 110 moves closer to heart 4: (1) energy level of the P-wave increases until the tip of catheter 110 with sensor 120 has reached a point closest to heart 4 when there is a sudden drop in energy; (2) a level of P-wave slope changes from positive to negative (or vice-versa, since slope change is relative); and (3) P-wave to R-wave amplitude ratio also increases until heart 4 is reached when it suddenly declines. Using these 3 parameters, and their inflections, as thresholds, may display or otherwise alert users of an accurate indication that tip of catheter 110 has reached a point closest to heart 4 and is accurately placed.

Example embodiment ECG system 100 may use multiple parameters of an ECG signal detected from sensor 120 for accurate navigation and positioning. The use of direct heartbeat electrical information, and further multiple independent parameters of the same, in system 100 may improve navigation and location of the tip of catheter 110 during central venous access. This does not require, and may be simpler than, X-ray, ultrasound, or fluoroscopy, and as such may be a cost-effective alternative to such radiological controls

It will be appreciated by one skilled in the art that example embodiments may be varied through routine experimentation and without further inventive activity. For example, although specific types of catheters and visual displays are used in some example embodiments, it is understood that other catheters or venous instrumentation, and other outputs, such as auditory alerts or tactile feedback, can be used in functionally equivalent procedures. Variations are not to be regarded as departure from the spirit and scope of the exemplary embodiments, and all such modifications as would be obvious to one skilled in the art are intended to be included within the scope of the following claims.

What is claimed is:

1. A system for positioning instrumentation with respect to the heart, the system comprising:
an instrument configured to navigate inside a blood vessel of the cardiovascular system;
a sensor attached to a distal end of the instrument, wherein the sensor is configured to detect electrical signals from the heart; and
a receiver communicatively connected to the sensor and a hardware processor, wherein the receiver and processor are configured to,
determine a P-wave and an R-wave of the heart from the electrical signals, determine at least one of an energy of the P-wave,
determine a ratio of an amplitude of the P-wave and the R-wave,
determine a slope of the P-wave, and
provide positioning information of the instrument based on at least one of the energy of the P-wave, the ratio of the amplitude of the P-wave and the R-wave, and the slope of the P-wave.

2. The system of claim 1, wherein the instrument is a catheter, and wherein the sensor is at a distal tip of the catheter.

3. The system of claim 1, wherein the catheter is a peripherally-inserted central catheter or a central venous catheter.

4. The system of claim 1, wherein the receiver and processor are configured to determine when the electrical signals reach a threshold, and wherein the positioning information includes a visual or auditory alert when the threshold is reached.

5. The system of claim 1, wherein the positioning information is the energy of the P-wave over time.

6. The system of claim 1, wherein,
the receiver and processor are configured to determine when the electrical signals reach a threshold,
the threshold is at least one of the energy of the P-wave decreasing, a slope of the P-wave reversing, and the ratio of the amplitude of the P-wave and the R-wave decreasing, and
the positioning information includes an alert when the threshold is reached.

7. The system of claim 1, further comprising:
at least one of a display and a speaker providing the positioning information.

8. The system of claim 7, wherein the display and the speaker are configured to provide the positioning information as the energy of the P-wave over time.

9. The system of claim 7, wherein the display and the speaker are configured to provide the positioning information as the slope of the P-wave over time.

10. The system of claim 7 wherein the display and the speaker are configured to provide the positioning information as the ratio of the amplitude of the P-wave and the R-wave over time.

11. The system of claim 10, wherein,
the instrument is a peripherally-inserted central catheter or a central venous catheter,
the sensor is at a distal tip of the catheter,
the receiver and processor are configured to determine a slope of the P-wave,
the display and the speaker are configured to provide the positioning information as the slope of the P-wave over time,
the receiver and processor are configured to determine an energy of the P-wave, and
the display and the speaker are configured to provide the positioning information as the energy of the P-wave over time.

12. A method of positioning an instrument in a blood vessel of a cardiovascular system, the instrument including a sensor at a distal end of the instrument configured to detect electrical signals from the heart and communicatively connected to a receiver and a hardware processor configured to:
determine a P-wave and an R-wave of the heart from the electrical signals, determine at least one of an energy of the P-wave,
determine a ratio of an amplitude of the P-wave and the R-wave,
determine a slope of the P-wave, and
provide positioning information of the instrument, while the instrument is being inserted into the blood vessel, based on at least one of the energy of the P-wave, the ratio of the amplitude of the P-wave and the R-wave, and the slope of the P-wave.

13. The method of claim 12, wherein the instrument is a catheter, and wherein the sensor is at a distal tip of the catheter.

14. The method of claim 13, wherein the catheter is a peripherally-inserted central catheter or a central venous catheter.

15. The method of claim 12, wherein the providing the positioning information includes at least one of a displaying the information on a display and making auditory alerts over a speaker.

16. The method of claim 12, further comprising:
determining when the electrical signals reach a threshold, wherein the threshold is at least one of the energy of the P-wave decreasing, a slope of the P-wave reversing, and the ratio of the amplitude of the P-wave and the R-wave decreasing, and wherein the providing includes issuing an alert when the threshold is reached.

17. The method of claim 12, wherein the providing includes displaying the determined at least one of the energy of the P-wave, the ratio of the amplitude of the P-wave and the R-wave, and the slope of the P-wave over time in a visual chart on a display.

18. The method of claim 12, further comprising:
moving the instrument in the blood vessel based on the provided positioning information.

19. The method of claim 18, further comprising:
determining when the electrical signals reach a threshold, wherein the threshold is at least one of the energy of the P-wave decreasing, a slope of the P-wave reversing, and the ratio of the amplitude of the P-wave and the R-wave decreasing, and wherein the providing includes issuing an alert when the threshold is reached; and
stopping the moving when the threshold is reached.

20. The method of claim 12, wherein the instrument is a peripherally-inserted central catheter or a central venous catheter, and wherein the positioning information is a position of the instrument with respect to the heart.
